(19) United States
(12) Patent Application Publication
(10) Pub. No.: US 2025/0315819 A1
(43) Pub. Date: Oct. 9, 2025

(54) Integrated Hosted Solutions

(71) Applicant: Synchrony Bank, Stamford, CT (US)

(72) Inventors: Kristin Schriver, Stamford, CT (US); Virginia Hall, Stamford, CT (US); Osvaldo Monge, Stamford, CT (US); Clarinda Gurr, Stamford, CT (US); Kendra Larsen, Stamford, CT (US)

(21) Appl. No.: 19/170,166

(22) Filed: Apr. 4, 2025

Related U.S. Application Data
(60) Provisional application No. 63/575,337, filed on Apr. 5, 2024.

(51) Int. Cl. G06Q 20/38; G06Q 20/40; G16H 10/60

(52) U.S. Cl. CPC G06Q 20/382; G06Q 20/407; G16H 10/60

(57) ABSTRACT

Methods are described herein for facilitating one or more requests using an integrated service. In some examples, the method may include receiving a request to initiate a secured transaction associated with a patient account. The method may also include generating one or more inquiries pertaining to the secured transaction. The method may also include transmitting, a data request to a secured database for patient data associated with the secured transaction and the patient account. The method may also include receiving the patient data. The method may also include populating a portion of the one or more inquiries by generating a comparison of the one or more inquiries to the patient data and by identifying at least one matching inquiry based on the comparison. The method may also include facilitating presentation of the one or more inquiries on a user interface of a display device.

### CROSS-REFERENCE TO RELATED APPLICATIONS

This application claims priority to and the benefit of U.S. Provisional Application No. 63/575,337, filed Apr. 5, 2024, titled “INTEGRATED HOSTED SOLUTIONS,” the disclosure of which is hereby incorporated by reference in its entirety.

### TECHNICAL FIELD

This disclosure relates generally to data transmission, and more particularly to facilitating and optimizing transmission of requests from a user device using an integrated service associated with a provider management software.

### BACKGROUND

Healthcare providers, clinicians, health clinics, hospitals, specialty practices, dental offices, or any other related healthcare-associated provider may utilize a practice management software to facilitate transactions between the healthcare provider and a patient, organize patient data, schedule appointments, and/or any other administrative task necessary to conduct business as a healthcare provider. The practice management software may be operated by a third-party entity, but customized for the healthcare provider. In some examples, the practice management software is not conducive to managing complicated transactions (e.g., payment with credit, payment by an individual other than the patient, etc.) and requires additional effort by the healthcare provider to complete the complicated transaction. Additionally, in some examples, the practice management software may not include a method of managing transactions at all, requiring the healthcare provider to identify a secondary software that must be cooperative with the practice management software.

### SUMMARY

Methods are described herein for facilitating and optimizing transmission of one or more requests from a user device using an integrated service associated with an independent software vendor (ISV). In some examples, the method may include receiving a request to initiate a secured transaction associated with a patient account, where the request is received by a server associated with an integrated service, and where a plurality of instances of the request are performed by the server simultaneously. The method may also include generating one or more inquiries pertaining to the secured transaction, where the one or more inquiries are generated using the request, and where the one or more inquiries are requests for information used to process the secured transaction. The method may also include transmitting, using a first custom application programming interface (API) a data request to a secured database for patient data associated with the secured transaction and the patient account. The method may also include receiving, from the secured database using the first custom API, the patient data. The patient data may include secure private data associated with the secured transaction. The method may also include populating a portion of the one or more inquiries by generating a comparison of the one or more inquiries to the patient data and by identifying at least one matching inquiry based on the comparison. The at least one matching inquiry may indicate that an element of patient data corresponds to an inquiry from the one or more inquiries. The method may also include facilitating presentation of the one or more inquiries on a user interface of a display device, wherein the portion of the one or more inquiries are distinguished upon presentation by populating the portion of the one or more inquiries with respective elements of patient data.

Systems are described herein for facilitating and optimizing transmission of one or more requests from a user device using an integrated service. The systems may comprise one or more processors and a memory storing instructions that, as a result of being executed by the one or more processors, cause the system to perform any of the aforementioned methods.

Non-transitory computer-readable storage media are described herein that store instructions therein that, as a result of being executed by one or more processors, cause the one or more processors to perform the any of the aforementioned methods.

Various embodiments of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which can be exhibited by some embodiments and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Without intent to limit the scope of the disclosure, examples of instruments, apparatus, methods, and their related results according to the embodiments of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

### BRIEF DESCRIPTION OF THE DRAWINGS

To easily identify the discussion of any particular element or act, the most significant digit or digits in a reference number refer to the figure number in which that element is first introduced.

FIG. 1 illustrates an example block diagram for implementing an integrated service according to some aspects of the present disclosure.

FIG. 2 illustrates an example process for data transmission between one or more components associated with the integrated service according to some aspects of the present disclosure.

FIG. 3 illustrates an example process for data transmission between one or more components associated with the integrated service according to some aspects of the present disclosure.

FIG. 4 illustrates an example flowchart for implementing an integrated service according to some aspects of the present disclosure.

FIG. 5 illustrates an example computing device capable of executing the integrated service and any other associated components according to some aspects of the present disclosure.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Disclosed embodiments may provide a framework through which one or more machine learning algorithms and programmatic logic are implemented to dynamically, and in real-time, process incoming invoices and corresponding claims as these invoices are received to identify one or more conditions for which treatment was provided. Based on these identified one or more conditions, as well as any known historical data corresponding to the claimant, adjudication of the invoices and corresponding claims may be performed.

The present disclosure includes a system and method for facilitating transactions with an integrated service associated with an independent software vendor (ISV), such as a third-party practice management software. An ISV may be a software, either installed on a local server, accessed via the Internet, accessible via cloud storage, any combination thereof, or the like, that a software user (e.g., a healthcare provider and/or healthcare office) may access to manage operations. For example, the ISV may be used to manage patient data, schedule and track appointments, communicate with patients, update medical records of patients, facilitate transactions, any combination thereof, or the like. In some examples, the ISV may be hosted on a central server and/or cloud server and one or more devices, offices, departments, or the like, may access instances of the ISV accordingly. The integrated service may be incorporated into the ISV but may be managed by a central controller not associated with the ISV. For example, to conduct updates on the integrated service, the central controller may initiate the updates, and the updates may be automatically implemented in the integrated service without involvement from the ISV or an affiliate third-party.

An indication of the integrated service may appear on a user interface (e.g., a graphical user interface (GUI), a web page, an application, etc.) associated with the ISV. The indication may be in the form of a button, a link, a menu option, an icon, a title, any combination thereof, or the like. An administrator associated with the healthcare provider may select, “click,” touch (on a touchscreen interface), or the like, the indication to access the integrated service. Once the integrated service has been selected by the administrator on a device, the device may be redirected to a second GUI, webpage, application, or the like, associated with the integrated service. On the integrated service GUI, the administrator may navigate to achieve one or more goals, including facilitating a transaction, conducting a return, applying for a loan, communicating with a patient, any combination thereof, or the like. The administrator, depending on the goal, may be prompted to fill in information corresponding to the patient, healthcare provider, service, transaction, any combination thereof, or the like. For example, if the patient wishes to apply for a loan to pay for medical services, the administrator may be prompted to input the patient's name, contact information, social security number, income information (e.g., paystubs), data necessary to perform a credit check, any combination thereof, or the like.

The integrated service and the ISV may be incorporated such that the integrated service may access one or more secured databases associated with the ISV. For example, the ISV may manage a database (stored locally, on a cloud-based storage platform, any combination thereof, or the like) that may contain privileged health-related information associated with one or more patients. This may include medical history, demographic information, appointment history, social security numbers, contact information, payment information, and the like. The integrated service, through an encrypted and/or secured connection, may retrieve data from the one or more secured databases. Thus, when an administrator is attempting to facilitate a transaction on the integrated service, the integrated service may pre-populate one or more necessary fields with data from the one or more secured databases (e.g., name of patient, address of patient, social security number of patient, etc.), resulting in a more streamlined process for the patient, healthcare provider, and administrator.

The integrated service, once appropriate information has been received, may transmit a request to a transaction manager associated with the integrated service. The request may include a request to submit a payment, a request for a loan, a request to distribute a refund, any combination thereof, or the like. In response to the request, the transaction manager may process the request and transmit a response. For example, the transaction manager may notify the integrated service that the payment method was declined, the loan was approved, the refund has been initiated, any combination thereof, or the like. Subsequently, the integrated service may notify the ISV and/or the user device to “pull back” the data associated with the transaction (i.e., request data associated with the transaction, the request, the response from the transaction manager, etc.). The ISV may pull back the data and may update the one or more secured databases accordingly (e.g., add a transaction associated with a patient, add new billing information associated with a patient, update the appointment and payment history for a patient, etc.). The ISV may notify the administrator and/or the patient according to the response from the transaction manager. In some examples, the integrated service may notify the administrator and/or the patient.

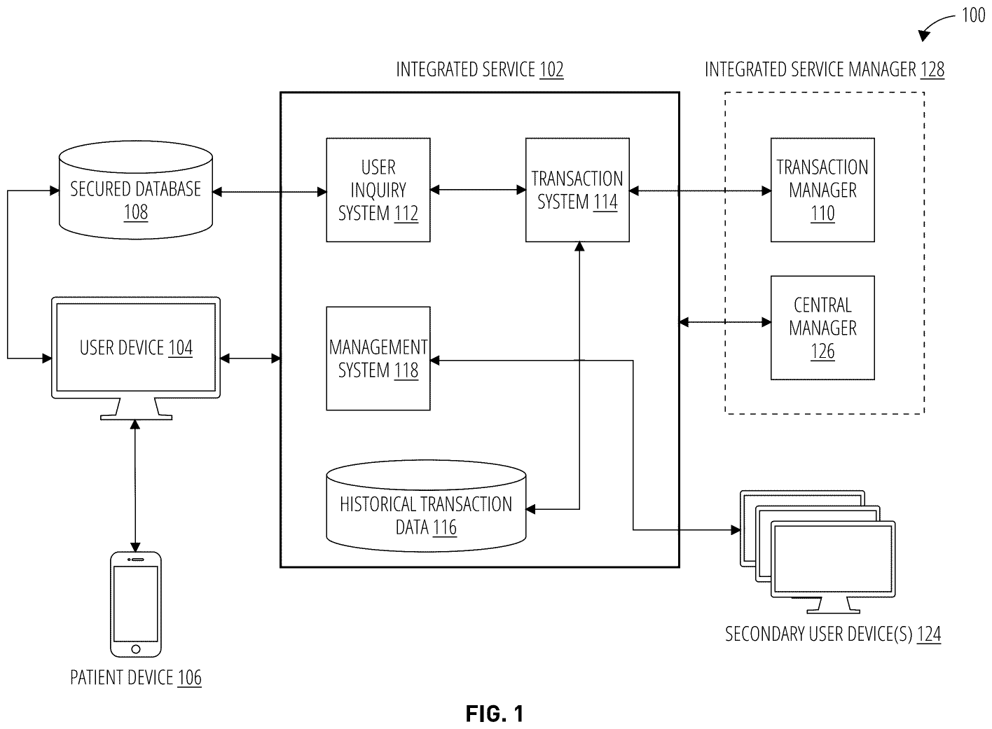

FIG. 1 illustrates an example block diagram for implementing an integrated service 102 according to some aspects of the present disclosure. The integrated service 102 may be a service implemented as a plug-in to a pre-existing third-party practice management software (ISV). The integrated service 102 may assist healthcare providers in facilitating transactions, reviewing patient files, viewing a transaction status, any combination thereof, or the like. System 100, as shown in FIG. 1, may represent one or more components necessary to implement integrated service 102 within an ISV. The ISV may be associated with user device 104, which may access the ISV by downloading software, via the Internet on a subscription or otherwise, at an instance provided by a cloud provider, any combination thereof, or the like. User device 104 may be a computing device associated with a healthcare provider and/or healthcare office. For example, user device 104 may be a laptop, desktop computer, smartphone, tablet, or the like. Integrated service 102 may be installed and/or linked within the ISV as an “add-in,” such that user device 104 may access integrated service 102 through the ISV. For example, integrated service 102 may appear as a “button,” link, tool, icon, or the like on a GUI associated with the ISV. Using the integrated service 102 button, a user associated with user device 104 may initiate integrated service 102 and begin operating integrated service 102 to streamline one or more otherwise tedious services, such as facilitating payments, applying for loans, granting refunds, etc.

User device 104 may request to launch integrated service 102 by touching, clicking, or otherwise selecting the integrated service 102 button associated with the ISV. In some examples, the integrated service 102 button may be in different locations or represented in a different manner depending on the variety of ISV and/or GUI associated with the ISV. For example, a first third-party entity operating a first ISV may incorporate integrated service 102 through the addition of a “button” at the top of a GUI, while a second third-party entity operating a second ISV may incorporate integrated service 102 through the addition of a banner on the right-hand side of a GUI. The presentation of the integrated service 102 button on the ISV may not impact the functionality of integrated service 102. After user device 104 requests to launch integrated service 102, integrated service 102 may redirect user device 104 to a website, GUI, application, or the like configured to present integrated service 102 on user device 104. In some examples, the integrated service 102 GUI may be included within the ISV program, hosted on an Internet site, in a separate application, hosted on a cloud provider, any combination thereof, or the like. The integrated service 102 GUI may present one or more options to user device 104, such as “apply,” “purchase,” “refund,” “lookup,” or any other options that may be related to healthcare-related transactions. By clicking, touching, or otherwise selecting an option of the one or more options, user device 104 may be additionally redirected to alternative locations within the integrated service 102 GUI specially configured to process a type of request from user device 104.

Integrated service 102 may include one or more components, including user inquiry system 112, transaction system 114, management system 118, and historical transaction data 116. Each component may be implemented individually or in combination to operate integrated service 102. Integrated service 102 may communicate and transmit data with user device 104 using the components described herein, or other components associated with integrated service 102. Integrated service 102 may be hosted on a remote server, a cloud provider, on a local device, any combination thereof, or the like.

A receptionist, doctor, hygienist, nurse, office administrator, or any other individual associated with a healthcare practice may access integrated service 102 on user device 104 to conduct some sort of action on behalf of a patient. For example, a patient may wish to make a payment for a healthcare service provided by the healthcare practice. When user device 104 indicates a request to complete an action using integrated service 102 (e.g., apply for a loan, make a purchase, refund a customer, etc.), the request may prompt user inquiry system 112 to generate one or more user inquiries necessary to complete the action. The one or more user inquiries may be based on prior transactions, requirements by transaction manager 110 (e.g., a bank facilitating a loan may require particular information from the loan applicant, a payment processing entity may require particular information from a payor to complete a payment, etc.), generated by the healthcare practice (e.g., generated by an accounting department of a hospital, etc.), any combination thereof, or the like.

In some examples, the one or more user inquiries may be generated by a machine-learning algorithm trained to predict information necessary to complete a particular action. The machine-learning algorithm may receive the request to complete the action and may be trained to generate one or more user inquiries that may be related to the requested action. Examples of machine-learning algorithms include algorithms such as k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, linear classification, artificial neural networks, anomaly detection, and such. More generally, machine learning or artificial intelligence methods may include regression analysis, dimensionality reduction, meta-learning, reinforcement learning, deep learning, and other such algorithms and/or methods. In some instances, the machine-learning algorithm may be trained using training data received and/or derived from one or more actions and respective user inquiries associated with the one or more actions. In some examples, the first machine-learning model may be trained using training data received and/or derived from historical data (e.g., historical transaction data 116) associated with prior transactions associated with integrated service 102. In some instances, the first machine-learning model may be trained using data associated with other implementations of integrated service 102 (e.g., an instance of integrated service 102 associated with another office, enterprise, or organization). The machine-learning algorithm can be trained using supervised training, supervised training, semi-supervised training, reinforcement training, combinations thereof, or the like.

For example, the machine-learning algorithm may be trained using transfer learning. Transfer learning is a technique in machine learning where a machine-learning algorithm initially trained to solve a particular task is used as the starting point for a different task. Transfer learning can be useful when the second task is somewhat similar to the first task, or when there is limited training data available for the second task. For example, a machine-learning algorithm initially trained to generate one or more user inquiries may be further trained to generate one or more user inquiries associated with a particular action (e.g., “make a payment”). In some instances, integrated service 102 may access a pre-trained model and “fine-tune” the pre-trained model by training the pre-trained model on a second training dataset. The second training dataset can include training data that includes user inquiries that are labeled with one or more possible actions. To further fine-tune the machine-learning algorithm, integrated service 102 reconfigures the machine-learning algorithm to include additional hidden and/or output layers to recognize information necessary to complete a particular action. In some instances, fine-tuning the pre-trained model includes unfreezing some of the layers of the pre-trained model and training them on the new training dataset. The number of layers that are unfrozen can depend on the size of the new dataset and how similar it is to the original dataset. For example, the fine-tuning of the first machine-learning model can include freezing the weights of the first machine-learning model, to train the first machine-learning model to predict user inquiries necessary to complete a particular action. Then, the weights can be unfrozen such that the first machine-learning model can be trained to improve accuracy of the user inquiries. By improving accuracy of the machine-learning algorithm, this reduces the processing load on computers associated with integrated service 102 by reducing the number of iterations necessary to gather information necessary to complete an action. Further, this reduces the likelihood that irrelevant information is gathered and processed, thereby reducing a processing load on the system.

The one or more user inquiries may include prompts requesting information to be input by user device 104. User device 104 may input data through one or more input devices, including, but not limited to, a keyboard, a mouse, a microphone, and/or a touchscreen interface. For example, the individual associated with the healthcare practice may type in responses to the one or more user inquiries on the keyboard of a desktop computer. The prompts may include requests for patient data, transaction data, healthcare provider data, appointment data, payment method data, record locators, any combination thereof, or the like. For example, to complete payment for a healthcare appointment, integrated service 102 may request a patient name or identification number, payment method, appointment type, a billing address, and an email to send a receipt. The individual associated with the healthcare practice may relay the one or more user inquiries to the patient and input responses on behalf of the patient. For example, a receptionist may ask for the patient's credit card information and may input the credit card information into integrated service 102 using user device 104 on behalf of the patient.

In some examples, integrated service 102 may determine to transmit the one or more user inquiries to patient device 106. The determination to transmit the one or more user inquiries may be performed by the machine-learning algorithm. The machine-learning algorithm may be further trained to determine whether or not to immediately transmit the one or more user inquiries to patient device 106 (e.g., without presenting the user inquiries to user device 104). A third training dataset may include historical data associated with historical user inquiries associated with one or more user accounts. For example, the machine-learning algorithm may determine that historically, a first user calls to pay bills associated with a user account of the first user. Therefore, the machine-learning algorithm may automatically transmit the one or more user inquiries to patient device 106 based on historical data associated with the first user. In some other examples, the machine-learning algorithm may automatically transmit the one or more user inquiries to patient device 106 based on historical data associated with users similarly situated to the first user. For example, if the first user is a minor and is unlikely to pay a medical bill personally, the machine-learning algorithm may automatically transmit the one or more user inquiries to patient device 106 based on historical data associated with other users that are also minors. In some examples, the one or more user inquiries may be transmitted to patient device 106 after receiving user input from user device 104 (e.g., at a touchscreen, at a keyboard, with a mouse click, any combination thereof, or the like.) In some other examples, the one or more user inquiries may be transmitted to patient device 106 after a duration of time (e.g., 30 seconds pass without input from user device 104).

For example, if the patient wishes to fill out the one or more user inquiries discreetly (e.g., without relaying answers to a receptionist, etc.), the patient may request the one or more user inquiries be sent to her cell phone so the patient can input responses privately. In some other examples, this enables a parent of a minor to input payment information, confidential identification information, or otherwise as a response to one or more user inquiries without entrusting this information and/or data to a minor child attending a healthcare appointment without the parent (e.g., a 17-year-old child attending a dental cleaning without an accompanying parent, a parent is waiting in the car, etc.). The transmitted inquiries may include all of the one or more user inquiries or a select few of the one or more user inquiries. For example, user device 104 may complete requests associated with the appointment and/or the healthcare provider and may transmit requests associated with payment information to patient device 106. All or some of the one or more user inquiries may be transmitted to patient device 106 via email, text message, web link, any combination thereof, or the like. For example, user device 104 may send an email containing an embedded link to patient device 106, where the embedded link redirects patient device 106 to a website containing the one or more user inquiries transmitted to patient device 106 by user device 104. In some other examples, patient device 106 may receive SMS/text message/mobile message prompts containing a request. For example, patient device 106 may receive a text message containing the question “What is the patient's date of birth?” Patient device 106 may input a response to the question (e.g., “Apr. 10, 2001”) using one or more input methods, including a touchscreen interface, a microphone, etc.

In some examples, to expedite responding to requests of the one or more user inquiries, secured database 108 may be accessed to generate responses to some, none, or all of the one or more user inquiries generated by user inquiry system 112. The one or more user inquiries generated by user inquiry system 112 may be automatically populated with data stored within secured database 108 without input required from user device 104 or patient device 106. For example, after user device 104 transmits the request to complete an action and user inquiry system 112 generates one or more user inquiries necessary to complete the action, secured database 108 may be queried to identify data pertinent to the one or more user inquiries. In some examples, a minimal amount of input may be required from user device 104 in order to identify pertinent information (e.g., integrated service 102 may prompt user device 104 for a patient name or birthday). In some other examples, using cached data, cookies, machine-learning, any combination thereof, or the like, integrated service 102 may identify contextually relevant data in order to identify pertinent information within secured database 108. For example, the machine-learning algorithm may be further trained to identify contextually relevant data using a fourth training dataset that includes historical data (e.g., historical transaction data 116) that identifies contextually relevant data associated with an action a customer identifier. For example, the machine-learning algorithm may automatically identify contextually relevant data within secured database 108 based on a customer name and an action.

As another example, if the ISV software had navigated to a particular patient profile, appointment, medical history, or otherwise, shortly before initiating integrated service 102, integrated service 102 may identify data related to the particular patient profile, appointment, or medical history and use that data to identify pertinent information with secured database 108. The pre-population of responses to one or more user inquiries may assist the individual associated with the healthcare practice and/or the patient with responding to the one or more user inquiries. For example, upon presentation of the one or more user inquiries via user device 104 to the individual associated with the healthcare practice, the individual may observe that some inquiries have already been “filled in” or “answered” by the data gathered from secured database 108. In some other examples, the “filled in” or “answered” inquiries may be omitted from the presentation of the one or more user inquiries via user device 104 (e.g., the “filled in” inquiries are not presented on a user interface associated with user device 104) and/or transmission of the one or more user inquiries to patient device 106 (e.g., the “filled in” inquiries are not transmitted to patient device 106). As a result, this reduces processing required to obtain data associated with the one or more user inquiries. Further, this procedure saves individuals associated with the transaction (e.g., the healthcare provider, the patient, the office administrator facilitating the transaction at user device 104, etc.) valuable time and increases office efficiency. Additionally, this procedure may increase accuracy of data by avoiding user input mistakes.

Secured database 108 may store data related to patients, appointments, payment information, healthcare providers, medical history, any combination thereof, or the like. In some examples, secured database 108 may be implemented by integrated service 102. The data may be stored according to a custom data structure and/or storage mechanism. The custom data structure and/or storage mechanism may be dependent on a search algorithm utilized by integrated service 102 to identify pertinent information within secured database 108 (e.g., linear search, binary search, hashing, sequential search, index-based search, graph search algorithms, distributed search algorithms, etc.). For example, secured database 108 may utilize arrays, linked lists, stacks, queues, trees, graphs, hash tables, heaps, skip lists, any combination thereof, or the like, to store data within secured database 108. The custom data structure may be implemented in a relational database, NoSQL database, in-memory database, graph database, object-oriented database, cloud-based database, any combination thereof, or the like. For examples, secured database 108 may store a patient's name, medical history, most recent appointment information, account balance, and/or any other relevant data in association with a patient ID number (e.g., a social security number, an ID number assigned by the healthcare provider or the ISV, etc.). Secured database 108 may be secured using one or more mechanisms of security, including, but not limited to, encryption (e.g., SSL/TLS, RSA, hash, etc.), authentication, access control, inference control, flow control, etc. Secured database 108 may receive the data from the ISV, the integrated service 102, user device 104, an external data source (e.g., a secondary cloud database source, local memory, another database, etc.), any combination thereof, or the like. Data stored within secured database 108 may be “tagged” or given an alternative form of identifier to provide ease of search. For example, payment information associated with a particular patient may be stored with the tag “payment,” “credit card,” “payment method,” along with the name or reference ID of the particular patient, to quickly identify and satisfy a request of the one or more user inquiries for payment information.

User inquiry system 112, prior to transmitting the one or more user inquiries to user device 104 and/or patient device 106 containing requests for data, may access secured database 108 to determine which, if any, of the one or more user inquiries may be satisfied using data stored in secured database 108. For example, user inquiry system 112, using an identifier associated with the request to complete an action (e.g., a patient ID number, an appointment date and time, a patient name, a patient birth date, etc.) may query secured database 108 for relevant data using one or more search algorithms (e.g., linear search, binary search, hashing, sequential search, index-based search, graph search algorithms, distributed search algorithms, etc.). The query and subsequent communications may be encrypted, protected, and/or secured through one or more means to preserve the sensitive information stored within secured database 108. For example, user inquiry system 112 may query secured database 108 for data matching a specific patient ID number provided by user device 104 and/or the ISV, which may include a name, payment information, demographic information, income information, etc.

In some examples, user inquiry system 112 may also receive data from user device 104 and/or the ISV to satisfy one or more of the one or more user inquiries. The data may be transmitted through a secured communication channel (e.g., encryption, authentication, error detection, shielding, directional transmission, etc.). For example, the ISV may transmit data pertaining to a particular appointment (e.g., scheduled time, duration, procedures performed, etc.), a patient (e.g., name, age, etc.), healthcare provider (e.g., name, charge rate, etc.), etc. This data, in lieu of or in addition to data gathered from other sources (e.g., secured database 108, user device 104, patient device 106, etc.), may be gathered to satisfy requests of the one or more user inquiries.

User inquiry system 112 may attempt to satisfy as many of the one or more user inquiries as possible using alternative sources (e.g., secured database 108, the ISV, etc.) prior to transmitting the remaining requests of the one or more user inquiries to user device 104 for manual entry or transmission to patient device 106 for manual entry. In some examples, the requests satisfied by the alternative sources may still be transmitted to user device 104 and/or patient device 106, but may show as “complete” or may display the data provided by the alternative source and provide an opportunity for user device 104 and/or patient device 106 to manually modify the pre-populated data. For example, one or more user inquiries requesting a patient name, birthday, and Social Security number may be prepopulated and “filled out” using data from secured database 108 and may be displayed as “filled out” when displayed via user device 104 and/or patient device 106. However, user device 104 and/or patient device 106 may include capabilities to modify the prepopulated data if the patient's name, birthday, and/or Social Security number are incorrect. In some examples, if the prepopulated data is modified by user device 104 and/or patient device 106, the modified data may be transmitted to secured database 108 and the data stored within secured database 108 may be updated (e.g., updating expired credit card information with new credit card information).

User device 104 and/or patient device 106 may submit responses to the one or more user inquiries to user inquiry system 112. User inquiry system 112 may further transmit the responses to transaction system 114, which may cross-reference the responses with historical transaction data 116 to determine the accuracy of the data provided. For example, transaction system 114 may detect a disparity between an income provided in a prior transaction and an income provided in a current transaction. This disparity may be associated with a name, an income, an identifier (e.g., Social Security Number, customer identifier, invoice number, etc.), a medical diagnosis, a date of a recent appointment, a birth date, an age, a weight, a height, a credit card number, a bank account and/or routing number, a debit card number, an email, a phone number, medical history information, any combination thereof, or the like. In some examples, transaction system 114 may determine whether to notify user device 104 and/or patient device 106 of the disparity. For example, the disparity may not materially impact the treatment and payment processing of a patient associated with the one or more user inquiries (e.g., weight, date of recent appointment). Thus, in some examples, transaction system 114 may flag the disparity for future identification (e.g., using a tag and/or metadata) and may not notify user device 104 and/or patient device 106 of the disparity. However, in some other examples, transaction system 114 may notify user device 104 of the disparity. In some examples, depending on the disparity detected by transaction system 114, transaction system 114 may reject the responses to the one or more user inquiries and return the one or more user inquiries to the user inquiry system 112 to be inspected and/or edited by user device 104 and/or patient device 106. For example, if a credit card number, phone number, address, or any other response is found to be invalid by transaction system 114 (e.g., by cross-referencing with an external third-party database, identifying a phone number response has an incorrect amount of digits, etc.), transaction system 114 may reject the responses to the one or more user inquiries and may notify user device 104 of the rejection and display at least the incorrect response via user device 104 for correction. In some examples, the rejection of the responses may be used to further train the machine-learning algorithm.

Transaction system 114 may store the responses to the one or more user inquiries in association with the current transaction in historical transaction data 116. Historical transaction data 116 may be accessed by integrated service 102 and/or other associated components (e.g., user inquiry system 112, transaction system 114, management system 118, user device 104) to access data related to prior transactions conducted by integrated service 102. In some examples, historical transaction data 116 may periodically purge data according to criteria specified by integrated service 102, user device 104, the ISV, any combination thereof, or the like. The data may be purged according to transaction date (e.g., transactions older than five years), storage capacity (e.g., oldest transactions are purged first when historical transaction data 116 reaches a certain size), transaction type (e.g., cash transactions are purged, while credit card transactions are kept), status (e.g., approved transactions are purged, while pending transactions are kept), any combination thereof, or the like.

Transaction system 114, once the data provided in response to the one or more user inquiries has been confirmed and approved by transaction system 114, may transmit the request and the corresponding responses to the one or more user inquiries to transaction manager 110. Transaction manager 110 may be located on a remote server and may be associated with a third party. For example, transaction manager 110 may be affiliated with a bank, loan manager, a point-of-sale service, integrated service manager 128, or the like. In some examples, integrated service manager 128 may be included within the functionality described in system 100. Integrated service manager 128 may be associated with integrated service 102 and may contribute to the functionality of integrated service 102 by providing updates, managing configurations, installing software, providing IT support, enforcing changes throughout a network of user devices (e.g., user device 104 and secondary user device(s) 124), any combination thereof. Integrated service manager 128 may be hosted in a location that may be the same or different as integrated service 102, user device 104, etc. For example, integrated service manager 128 may be hosted on a remote server that may be able to connect to integrated service 102.

In some examples, transaction manager 110 may identify one or more issues associated with the request and the corresponding responses. While transaction system 114 locally verifies that the provided data is accurate, transaction manager 110 verifies that the provided data is usable and sufficient to perform the desired action. For example, transaction manager 110 may identify missing data (e.g., phone number, date of birth, email address, etc.), incorrect data (e.g., invalid or declined credit card number, invalid Social Security number, invalid banking information, etc.), unnecessary data (e.g., appointment information, entity-specific identifier, healthcare provider information, etc.), any combination thereof, or the like, may be identified by transaction manager 110. In response to identifying the one or more issues, transaction manager 110 may transmit a notification to integrated service 102 associated with the one or more issues. The notification may include a request for more data, a request for revised data, a notification that excessive data was received, any combination thereof, or the like. Based on the notification, integrated service 102 may perform one or more actions to remedy the one or more issues indicated in the notification. For example, integrated service 102 may notify user device 104, may notify patient device 106, may generate additional user requests and transmit the additional user requests to patient device 106 and/or user device 104 (e.g., notify patient device 106 that a credit card number was invalid and request updated credit card information), any combination thereof, or the like. In some examples, the one or more issues may be used to update and/or further train the machine-learning algorithm. The one or more issues may be included in a training dataset that is used to refine the generation of the one or more user inquiries.

In addition to being comprised of transaction manager 110, integrated service manager 128 may also be comprised of central manager 126. Central manager 126 may be a central controller responsible for implementing software updates, notifications indicating hardware updates, assisting with the implementation of integrated service 102 into the ISV software, any combination thereof, or the like. Central manager 126 may be connected to multiple instances of integrated service 102 that may be associated with a different ISV and/or a different user device 104. For example, software changes to integrated service 102 may be implemented via central manager 126 at Hospital and Dentist Office simultaneously, even if Hospital and Dentist Office are two distinct, unaffiliated users of integrated service 102.

If an update is received from central manager 126 at integrated service 102, it may be processed through management system 118. Management system 118 may facilitate the synchronization of integrated service 102 between central manager 126, user device 104, and secondary user device(s) 124. For example, any settings associated with integrated service 102 that are implemented by user device 104 may be further implemented at secondary user device(s) 124 via management system 118. Additionally, notifications, updates, changes, or any other transmission received from central manager 126 may be implemented on integrated service 102 via management system 118. For example, if central manager 126 transmits an update to integrated service 102 that alters the color of the “button” displayed on the GUI of the ISV, management system 118 may implement that change on user device 104.

In some examples, a healthcare provider office may be associated with one or more “satellite” offices (i.e., affiliated offices or practices in another area of healthcare, another location, etc.). For example, user device 104 may be associated with a “home” office of Dental Practice, and secondary user device(s) 124 may be associated with one or more secondary locations of Dental Practice (e.g., Dental Practice #2 located in another town, Dental Practice #3 specializing in pediatric dental care, etc.). In some other examples, user device 104 and secondary user device(s) 124 may be associated with the same practice/location/office (e.g., user device 104 is the “primary” while secondary user device(s) 124 may be associated with individual rooms, stations, and/or offices within the location). In either scenario, changes implemented at the “home” or “primary” office on user device 104 pertaining to integrated service 102 may be implemented on local sessions of integrated service 102 hosted at secondary user device(s) 124. Secondary user device(s) 124 may have complete access to integrated service 102, secured database 108, and any other components described herein that are also accessible to user device 104. However, secondary user device(s) 124 may merely lack administrative authority and/or authentication to complete certain actions associated with integrated service 102, including, but not limited to, adding/removing data from secured database 108 and/or historical transaction data 116, initiate a particular type of transaction (e.g., a loan), make setting changes associated with integrated service 102 (e.g., notification preferences, time/location preferences, implementing recommended software updates, etc.), creating or altering patient profiles, any combination thereof, or the like.

Integrated service 102 may be utilized by more than one healthcare provider office at one time. The different healthcare provider offices may access distinct instances of integrated service 102 that may be customized to each individual healthcare provider office (e.g., historical transaction data 116 may be distinct for each individual healthcare provider office). However, integrated service 102 may be hosting many requests for actions at one time from user device 104 and/or other user devices with access to integrated service 102 (e.g., secondary user device(s) 124, user devices associated with other instances of integrated service 102, etc.). A server associated with integrated service 102 may be capable of managing multiple requests simultaneously and may process data in real-time.

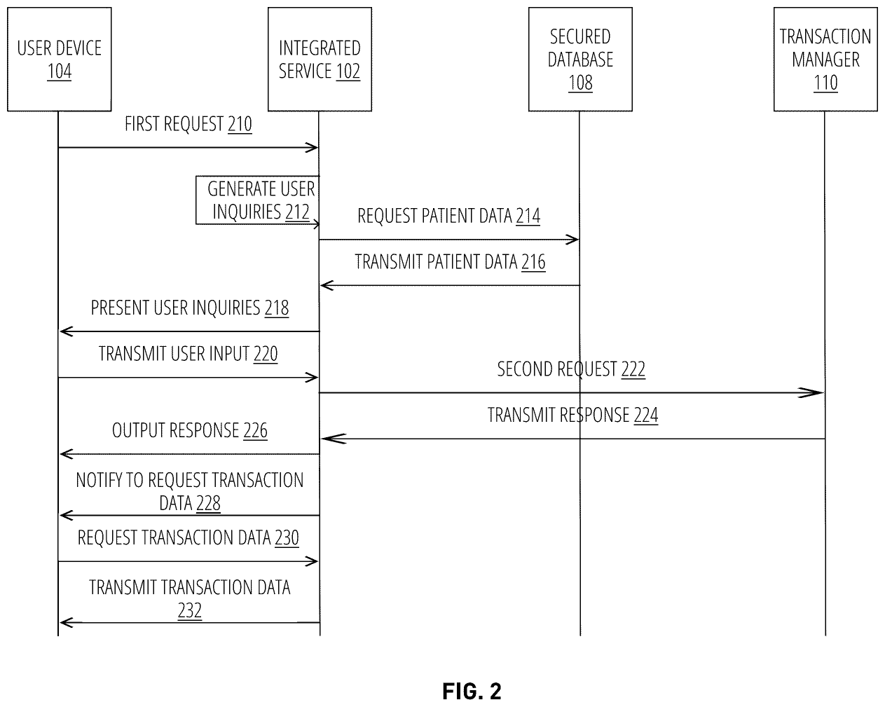

FIG. 2 illustrates an example process for data transmission between one or more components associated with the integrated service according to some aspects of the present disclosure. The process demonstrated in FIG. 2 is a mere example of how the components described in FIG. 1 and throughout the specification may transmit data. In some examples, the steps described herein may happen in a different order than described, the steps may occur simultaneously, some steps may be omitted or combined with another step, multiple instances of the same step may be occurring simultaneously, etc.

The user device 104 may be a user device similar to user device 104, as described in FIG. 1. It may be a personal computer, a tablet, a laptop, a desktop computer, a mobile phone, or the like. User device 104 may be associated with a particular healthcare provider, practice, office, location, any combination thereof, or the like. On user device 104, a practice management software (ISV) may be installed. The ISV is a third-party software or resource that a healthcare practice may utilize to manage appointments, billings, patient files, or other administrative aspects of the healthcare practice. In some examples, the ISV may be downloaded to user device 104, a website accessed on the Internet, hosted by a cloud provider, hosted at a remote server, any combination thereof, or the like. An individual associated with the healthcare practice, such as an administrator, may access user device 104 and the ISV.

Within the ISV may be a representation of integrated service 102. The representation may be demonstrated on a GUI associated with the ISV and integrated service 102 may be represented by a “button,” a menu option, a link, a symbol, any combination thereof, or the like. To initiate integrated service 102, user device 104, using an input device (e.g., a keyboard, a mouse, a touch screen, a voice command, etc.) may select the representation of integrated service 102 to launch integrated service 102. Upon the launch of integrated service 102, user device 104 may be redirected to a custom GUI associated with integrated service 102, which may be hosted within the ISV software, at a website, at a cloud provider, at a remote server, any combination thereof, or the like.

At step 210, the user device 104 may transmit a first request to integrated service 102. The first requested may be transmitted at the custom GUI associated with integrated service 102 via the input device of user device 104. The first request may include a request to complete a particular action, such as completing a transaction, apply for credit, process a refund, lookup the status of a particular transaction, any combination thereof, or the like.

At step 212, integrated service 102 may generate one or more user inquiries based on the first request. The one or more user inquiries may include requests for information and/or data required to complete the first request, such as patient information, appointment information, payment information, any combination thereof, or the like. The one or more user inquiries may be dependent on the action associated with the first request. For example, the one or more user inquiries may differ between a request to apply for credit (e.g., financial information of the patient, patient Social Security number, etc.) and a request to lookup the status of a particular transaction (e.g., transaction date, transaction ID number, patient ID number, etc.).

At step 214, integrated service 102 may request patient data from secured database 108 that correspond to the one or more user inquiries. Secured database 108 may be queried for responses to the one or more user inquiries. For example, integrated service 102 may query secured database 108 for a patient's name, address, and phone number that have been requested as an inquiry of the one or more user inquiries. In some examples, integrated service 102 may provide some initial identifying data to identify pertinent information within secured database 108. For example, integrated service 102 may provide a patient ID number, a patient name, an appointment date and time, a type of service provided, any combination thereof, or the like. The initial identifying data may be used by a search algorithm (e.g., linear search, binary search, hashing, sequential search, index-based search, graph search algorithms, distributed search algorithms, etc.) to identify pertinent data within secured database 108 (e.g., a name associated with a patient ID number, payment information associated with a patient ID number, etc.).

Secured database 108 may be configured according to a custom data structure and/or storage mechanism. The custom data structure and/or storage mechanism may be dependent on the search algorithm used by integrated service 102. For example, secured database 108 may utilize arrays, linked lists, stacks, queues, trees, graphs, hash tables, heaps, skip lists, any combination thereof, or the like, to store data within secured database 108. The custom data structure may be implemented in a relational database, NoSQL database, in-memory database, graph database, object-oriented database, cloud-based database, any combination thereof, or the like. For examples, secured database 108 may store a patient's name, medical history, most recent appointment information, account balance, and/or any other relevant data in association with a patient ID number (e.g., a social security number, an ID number assigned by the healthcare provider or the ISV, etc.). Secured database 108 may be secured using one or more mechanisms of security, including, but not limited to, encryption (e.g., SSL/TLS, RSA, hash, etc.), authentication, access control, inference control, flow control, etc.

At step 216, secured database 108 may transmit the pertinent data identified by the search algorithm to integrated service 102. In some examples, the pertinent data may be presented as raw data to integrated service 102. Using an integrated logic mechanism, identifiers within the pertinent data (e.g., tags, labels, etc.), machine-learning, natural language processing, any combination thereof, or the like, integrated service 102 may match the pertinent data to the one or more user inquiries. For example, integrated service 102 may identify a string of digits and recognize that string of digits to be a credit card number, thereby matching an inquiry for “credit card number” with the string of digits.

At step 218, user device 104 may present the one or more user inquiries via user device 104. The one or more user inquiries may be displayed via the ISV system, a GUI associated with integrated service 102, a website, any combination thereof, or the like. The one or more user inquiries may be displayed as a series of requests for information that a user of user device 104 may “fill out” or “complete” using one or more input devices. In some examples, the presented one or more user inquiries may include inquiries that were matched with data obtained from secured database 108. In these examples, the matched inquiries may be presented via user device 104 but may be displayed as already “filled out” or “completed.” In some examples, the user may manually modify the data supplied to the matched inquiries (e.g., if there is a mistake in the stored data, if the data needs to be updated, etc.). For example, if secured database 108 transmits data associated with an expired credit card as a response to a user inquiry, the user of user device 104 may modify the response with updated credit card information.

At step 220, user device 104 may submit user input responsive to the one or more user inquiries to integrated service 102. Using one or more input devices (e.g., a keyboard, a mouse, a touchscreen, a microphone, etc.), user device 104 may respond to the one or more user inquiries and submit the responses to integrated service 102 for additional processing. In some examples, user device 104 may not respond to all of the one or more user inquiries for one or more reasons, including, but not limited to, a user inquiry being marked as “optional,” a user inquiry being prepopulated with data obtained from secured database 108 or another source (e.g., the ISV, a patient device, an external database, etc.), any combination thereof, or the like.

At step 222, integrated service 102 may transmit a second request to transaction manager 110. The second request may be transmitted via a custom API configured to transmit the second request to transaction manager 110. The second request may include the data collected responsive to the one or more user inquiries, the action associated with the first request, any other necessary data from integrated service 102 and/or the ISV, any combination thereof, or the like. The transaction manager 110 may be associated with integrated service 102, a third-party, a bank or other financial institution, a payment platform, a database, a custom API, any combination thereof, or the like. In some examples, transaction manager 110 may vary depending on the action associated with the first request. For example, integrated service 102 may transmit the second request to a bank if the patient is requesting a line of credit but may transmit the second request to a custom API associated with a database if the patient is requesting a status update of a transaction.

At step 224, transaction manager 110 may transmit a response associated with the second request to integrated service 102. The response may be transmitted via the same custom API generated to transmit the second request to transaction manager 110. The response may include an approval (e.g., a payment approval, a credit line approval, etc.), requested data (e.g., a status update associated with a transaction), a confirmation (e.g., a confirmation that a payment has been processed, a confirmation that a refund has been processed, etc.), any combination thereof, or the like. At step 226, integrated service 102 may transmit the response to user device 104. In some examples, the response may be transmitted via a custom API configured to output data to user device 104. The response may be displayed on a user interface of user device 104 in the form of a notification, a pop-up, a redirect (e.g., automatically redirecting a web browser to a “confirmation” page), an email (e.g., receiving a receipt or confirmation email), a document (e.g., a document containing status information regarding a transaction), an update to data within the ISV, any combination thereof, or the like. In some examples, user device 104 may forward the response to a patient device associated with the transaction (e.g., send a text or email confirmation to the patient).

At step 228, integrated service 102 may notify user device 104 to request transaction data associated with the first and/or second request. The transaction data may include some or all data pertaining to the transaction and/or action performed between user device 104, integrated service 102, and/or transaction manager 110. The transaction data may include, but is not limited to, patient data, payment confirmation information, payment data, refund data, transaction status information, any combination thereof, or the like. At step 230, user device 104 may request the transaction data from integrated service 102. At step 232, integrated service 102 may transmit the transaction data to user device 104. In some examples, this series of communications between user device 104 and integrated service 102 may be referenced as a “pullback.” The data received from the pullback by user device 104 may be stored in a secured database (e.g., secured database 108 and/or another database associated with user device 104 and/or the ISV), transmitted to the patient, used to update local data (e.g., update accounting data, update scheduling for a patient, etc.).

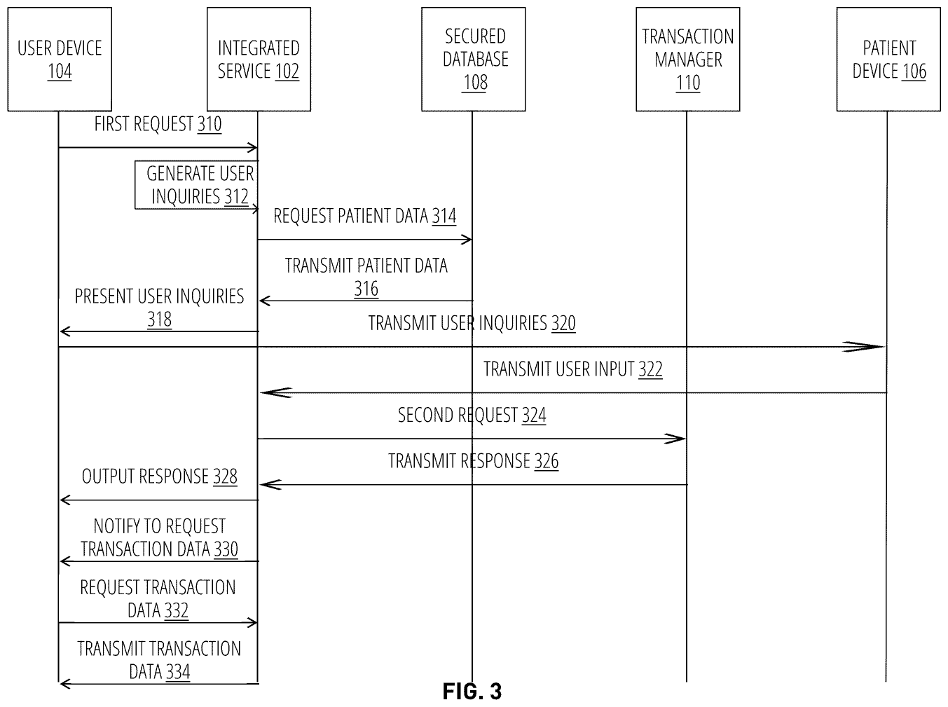

FIG. 3 illustrates an example process for data transmission between one or more components associated with the integrated service according to some aspects of the present disclosure. The process demonstrated in FIG. 3 is a mere example of how the components described in FIG. 1 and throughout the specification may transmit data. In some examples, the steps described herein may happen in a different order than described, the steps may occur simultaneously, some steps may be omitted or combined with another step, multiple instances of the same step may be occurring simultaneously, etc.

The user device 104 may be a user device similar to user device 104, as described in FIG. 1. It may be a personal computer, a tablet, a laptop, a desktop computer, a mobile phone, or the like. User device 104 may be associated with a particular healthcare provider, practice, office, location, any combination thereof, or the like. On user device 104, a practice management software (ISV) may be installed. The ISV is a third-party software or resource that a software user (e.g., a healthcare practice) may utilize to manage appointments, billings, patient files, or other administrative aspects of the healthcare practice. In some examples, the ISV may be downloaded to user device 104, a website accessed on the Internet, hosted by a cloud provider, hosted at a remote server, any combination thereof, or the like. An individual associated with the healthcare practice, such as an administrator, may access user device 104 and the ISV.

Within the ISV may be a representation of integrated service 102. The representation may be demonstrated on a GUI associated with the ISV and integrated service 102 may be represented by a “button,” a menu option, a link, a symbol, any combination thereof, or the like. To initiate integrated service 102, user device 104, using an input device (e.g., a keyboard, a mouse, a touch screen, a voice command, etc.) may select the representation of integrated service 102 to launch integrated service 102. Upon the launch of integrated service 102, user device 104 may be redirected to a custom GUI associated with integrated service 102, which may be hosted within the ISV software, at a website, at a cloud provider, at a remote server, any combination thereof, or the like.

At step 310, the user device 104 may transmit a first request to integrated service 102. The first requested may be transmitted at the custom API associated with integrated service 102 via the input device of user device 104. The first request may include a request to complete a particular action, such as completing a transaction, apply for credit, process a refund, lookup the status of a particular transaction, any combination thereof, or the like.

At step 312, integrated service 102 may generate one or more user inquiries based on the first request. The one or more user inquiries may include requests for information and/or data required to complete the first request, such as patient information, appointment information, payment information, any combination thereof, or the like. The one or more user inquiries may be dependent on the action associated with the first request. For example, the one or more user inquiries may differ between a request to apply for credit (e.g., financial information of the patient, patient Social Security number, etc.) and a request to lookup the status of a particular transaction (e.g., transaction date, transaction ID number, patient ID number, etc.).

At step 314, integrated service 102 may request patient data from secured database 108 that correspond to the one or more user inquiries. Secured database 108 may be queried for responses to the one or more user inquiries. For example, integrated service 102 may query secured database 108 for a patient's name, address, and phone number that have been requested as an inquiry of the one or more user inquiries. In some examples, integrated service 102 may provide some initial identifying data to identify pertinent information within secured database 108. For example, integrated service 102 may provide a patient ID number, a patient name, an appointment date and time, a type of service provided, any combination thereof, or the like. The initial identifying data may be used by a search algorithm (e.g., linear search, binary search, hashing, sequential search, index-based search, graph search algorithms, distributed search algorithms, etc.) to identify pertinent data within secured database 108 (e.g., a name associated with a patient ID number, payment information associated with a patient ID number, etc.).

Secured database 108 may be configured according to a custom data structure and/or storage mechanism. The custom data structure and/or storage mechanism may be dependent on the search algorithm used by integrated service 102. For example, secured database 108 may utilize arrays, linked lists, stacks, queues, trees, graphs, hash tables, heaps, skip lists, any combination thereof, or the like, to store data within secured database 108. The custom data structure may be implemented in a relational database, NoSQL database, in-memory database, graph database, object-oriented database, cloud-based database, any combination thereof, or the like. For examples, secured database 108 may store a patient's name, medical history, most recent appointment information, account balance, and/or any other relevant data in association with a patient ID number (e.g., a social security number, an ID number assigned by the healthcare provider or the ISV, etc.). Secured database 108 may be secured using one or more mechanisms of security, including, but not limited to, encryption (e.g., SSL/TLS, RSA, hash, etc.), authentication, access control, inference control, flow control, etc.

At step 316, secured database 108 may transmit the pertinent data identified by the search algorithm to integrated service 102. In some examples, the pertinent data may be presented as raw data to integrated service 102. Using an integrated logic mechanism, identifiers within the pertinent data (e.g., tags, labels, etc.), machine-learning, natural language processing, any combination thereof, or the like, integrated service 102 may match the pertinent data to the one or more user inquiries. For example, integrated service 102 may identify a string of digits and recognize that string of digits to be a credit card number, thereby matching an inquiry for “credit card number” with the string of digits.

At step 318, user device 104 may present the one or more user inquiries via user device 104. The one or more user inquiries may be displayed via the ISV system, a GUI associated with integrated service 102, a website, any combination thereof, or the like. The one or more user inquiries may be displayed as a series of requests for information that a user of user device 104 may “fill out” or “complete” using one or more input devices. In some examples, the presented one or more user inquiries may include inquiries that were matched with data obtained from secured database 108. In these examples, the matched inquiries may be presented via user device 104 but may be displayed as already “filled out” or “completed.” In some examples, the user may manually modify the data supplied to the matched inquiries (e.g., if there is a mistake in the stored data, if the data needs to be updated, etc.). For example, if secured database 108 transmits data associated with an expired credit card as a response to a user inquiry, the user of user device 104 may modify the response with updated credit card information.

At step 320, user device 104 may transmit the one or more user inquiries to patient device 106. Patient device 106 may be a personal device associated with a patient, such as a mobile phone, a smart phone, a laptop, a desktop, a tablet, a smartwatch, any combination thereof, or the like. The One or more user inquiries may be transmitted via SMS, Bluetooth, a cellular network, WiFi, any combination thereof, or the like. In some examples, patient device 106 may receive a notification (e.g., a text, an instant message, an email, an application notification, etc.) that may include a hyperlink to a website where patient device 106 may input responses to the one or more user inquiries. In some other examples, the one or more user inquiries may be transmitted individually to patient device 106. For example, patient device 106 may receive a text message asking, “what is your date of birth?” Patient device 106 may input a response to the text message, transmit the response, then receive a second text message that asks, “what is your mailing address?” This process may continue for some or all of the one or more user inquiries.

In some examples, not all of the one or more user inquiries may be transmitted to patient device 106. This may be determined according to a policy of the healthcare provider office, settings associated with integrated service 102, settings associated with transaction manager 110, any combination thereof, or the like. For example, one or more user inquiries that may pertain to personal information of the patient (e.g., demographic information, payment information, etc.) may be transmitted to patient device 106 for receipt and response by the patient, while one or more user inquiries that may pertain to the healthcare office (e.g., appointment information, expected cost, provider information, etc.) may not be transmitted to patient device 106. In such examples, user device 104 may response to the one or more user inquiries that may pertain to the healthcare office.

At step 322, patient device 106 may submit responses to the one or more user inquiries transmitted to patient device 106 to integrated service 102. Using one or more input devices (e.g., a keyboard, a mouse, a touchscreen, a microphone, etc.), patient device 106 may respond to the one or more user inquiries and submit the responses to integrated service 102 for additional processing. In some examples, patient device 106 may not respond to all of the one or more user inquiries for one or more reasons, including, but not limited to, a user inquiry being marked as “optional,” a user inquiry being prepopulated with data obtained from secured database 108 or another source (e.g., the ISV, a patient device, an external database, etc.), a user inquiry being responded to by user device 104, any combination thereof, or the like.

At step 324, integrated service 102 may transmit a second request to transaction manager 110. The second request may be transmitted via a custom API generated to transmit the second request to transaction manager 110. The second request may include the data collected responsive to the one or more user inquiries, the action associated with the first request, any other necessary data from integrated service 102 and/or the ISV, any combination thereof, or the like. The transaction manager 110 may be associated with integrated service 102, a third-party, a bank or other financial institution, a payment platform, a database, a custom GUI, any combination thereof, or the like. In some examples, transaction manager 110 may vary depending on the action associated with the first request. For example, integrated service 102 may transmit the second request to a bank if the patient is requesting a line of credit but may transmit the second request to a custom API associated with a database if the patient is requesting a status update of a transaction.

At step 326, transaction manager 110 may transmit a response associated with the second request to integrated service 102. The response may be transmitted via the same custom API generated to transmit the second request to transaction manager 110. The response may include an approval (e.g., a payment approval, a credit line approval, etc.), requested data (e.g., a status update associated with a transaction), a confirmation (e.g., a confirmation that a payment has been processed, a confirmation that a refund has been processed, etc.), any combination thereof, or the like. At step 328, integrated service 102 may transmit the response to user device 104. In some examples, the response may be transmitted via a custom API generated to output data to user device 104. The response may be displayed on a user interface of user device 104 in the form of a notification, a pop-up, a redirect (e.g., automatically redirecting a web browser to a “confirmation” page), an email (e.g., receiving a receipt or confirmation email), a document (e.g., a document containing status information regarding a transaction), an update to data within the ISV, any combination thereof, or the like. In some examples, user device 104 may forward the response to a patient device associated with the transaction (e.g., send a text or email confirmation to the patient).

At step 330, integrated service 102 may notify user device 104 to request transaction data associated with the first and/or second request. The transaction data may include some or all data pertaining to the transaction and/or action performed between user device 104, integrated service 102, and/or transaction manager 110. The transaction data may include, but is not limited to, patient data, payment confirmation information, payment data, refund data, transaction status information, any combination thereof, or the like. At step 332, user device 104 may request the transaction data from integrated service 102. At step 334, integrated service 102 may transmit the transaction data to user device 104. In some examples, this series of communications between user device 104 and integrated service 102 may be referenced as a “pullback.” The data received from the pullback by user device 104 may be stored in a secured database (e.g., secured database 108 and/or another database associated with user device 104 and/or the ISV), transmitted to the patient, used to update local data (e.g., update accounting data, update scheduling for a patient, etc.).

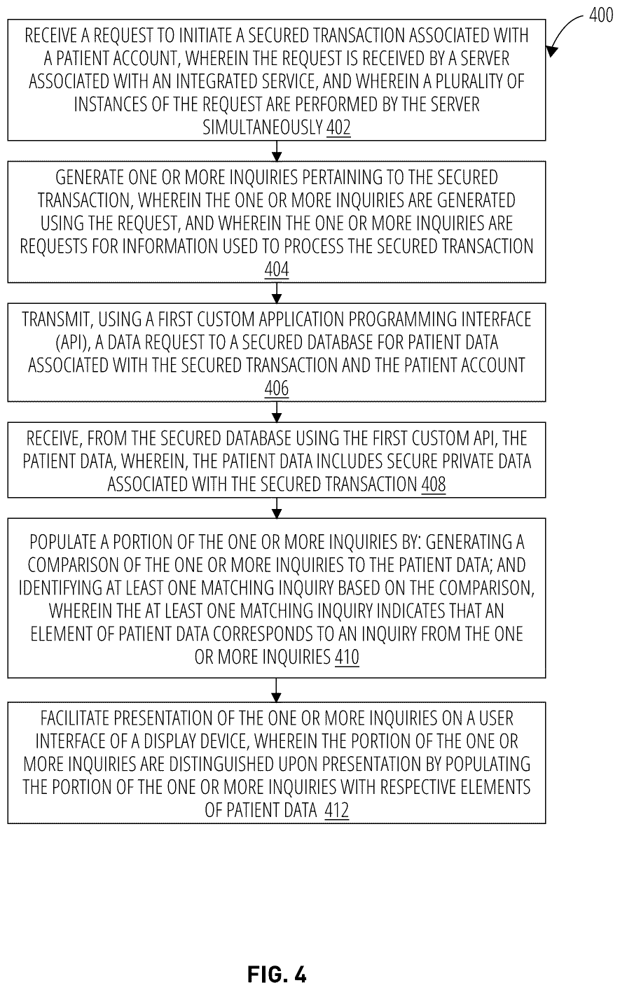

FIG. 4 illustrates an example flowchart for implementing an integrated service according to some aspects of the present disclosure. Although the example routine 400 depicts a particular sequence of operations, the sequence may be altered without departing from the scope of the present disclosure. For example, some of the operations depicted may be performed in parallel or in a different sequence that does not materially affect the function of the routine 400. In other examples, different components of an example device or system that implements the routine 400 may perform functions at substantially the same time or in a specific sequence.

At block 402, the method includes receiving a request to initiate a secured transaction associated with a patient account, wherein the request is received by a server associated with an integrated service, and wherein a plurality of instances of the request are performed by the server simultaneously. In some examples, the request may be transmitted by a user device associated with an independent software module (e.g., ISV or similar such software or programs operating on a computing device). The user device may be associated with a primary location, while one or more secondary user devices may be associated with one or more secondary locations (e.g., one central office with one or more satellite offices associated with the same healthcare practice). The user device may install one or more operational settings throughout a network of the one or more secondary user devices associated with the primary location, wherein the secondary user devices are unable to alter the one or more operational settings.

In some examples, the user device associated with the integrated service may receive one or more operational settings associated with the integrated service from a central server and/or central controller of the integrated service. The one or more operational settings may be software updates, updated hardware requirements, bug fixes, etc. The one or more operational settings may be automatically installed on the user device. In some examples, the one or more operational settings received from the central server and/or central controller may be distributed to the one or more secondary user devices by the user device, the central server, and/or central controller.

At block 404, the method includes generating one or more inquiries pertaining to the secured transaction, wherein the one or more inquiries are generated using the request, and wherein the one or more inquiries are requests for information used to process the secured transaction.

At block 406, the method includes transmitting, using a first custom application programming interface (API), a data request to a secured database for patient data associated with the secured transaction and the patient account.

At block 408, the method receiving, from the secured database using the first custom API, the patient data, wherein, the patient data includes secure private data associated with the secured transaction.

At block 410, the method includes populating a portion of the one or more inquiries by generating a comparison of the one or more inquiries to the patient data and identifying at least one matching inquiry based on the comparison, wherein the at least one matching inquiry indicates that an element of patient data corresponds to an inquiry from the one or more inquiries. Identifying the matching inquiry of the one or more offer inquiries is performed by a machine-learning model.

At block 412, the method facilitating presentation of the one or more inquiries on a user interface of a display device, wherein the portion of the one or more inquiries are distinguished upon presentation by populating the portion of the one or more inquiries with respective elements of patient data.

The presentation may be on a user interface of a user device. When the one or more offer inquires are received by the user device, the user device may transmit the one or more offer inquires to a patient device. When the one or more user inquiries are received by a patient device, the patient device may be associated with the patient data, the integrated service may receive patient input responsive to the one or more offer inquiries. In some examples, the integrated service may receive input responsive to the one or more offer inquiries from the user device. The integrated service may output a second request to a transaction manager, where the second request includes the input responsive to the one or more offer inquiries and the element of the patient data responsive to the matching inquiry. The integrated service may receive a response associated with the second request from the transaction manager. The integrated service may output the response using the second custom GUI to the user device. The second request may be a request for credit associated with the secured transaction. The second request may be a request to complete a payment associated with the secured transaction. In some examples, the integrated service may update the secured database with data pertaining to the secured transaction.

The first request may be transmitted by a plug-in operating from an independent software module (e.g., a plug-in associated with ISV software). The integrated service may transmit a notification to request transaction data associated with the secured transaction. When the independent software module (e.g., the ISV software installed on the user device) receives the notification, the independent software module transmits a transaction data request. The integrated service may then transmit data responsive to the transaction data request. When received by the independent software module, the independent software module updates one or more local databases associated with the user device with the data.

In some examples, the integrated service may output a third request to the transaction manager for a status associated with the secured transaction. In some examples, the integrated service may output a fourth request. When the fourth request is received by a transaction manager, the fourth request includes a completed transaction and patient data associated with the completed transaction. The integrated service may receive a response associated with the fourth request, where the response includes at least one of an approval or a denial of a refund associated with the completed transaction. The integrated service may output the response.

The integrated service may output a fifth request, wherein the fifth request is for patient data. The integrated service may receive patient data. When the patient data is received from the secured database, the patient data includes at least identifying information and historical completed transaction.

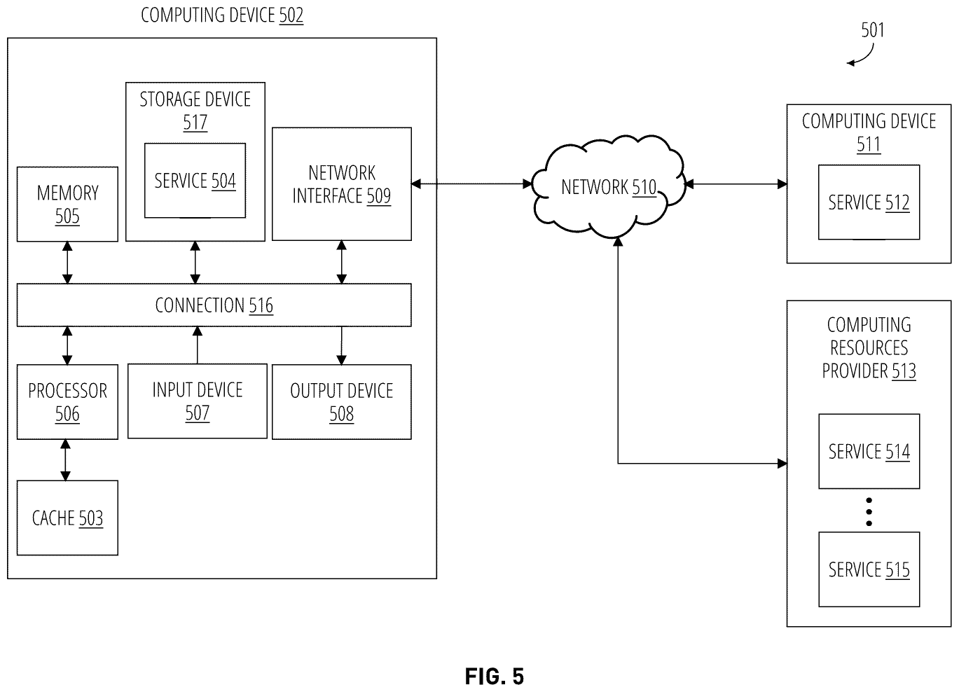

FIG. 5 illustrates an example computing device capable of executing the integrated service and any other associated components according to some aspects of the present disclosure. This may include a computing system architecture 501, including various components in electrical communication with each other. The example computing system architecture 501 illustrated in FIG. 5 includes a computing device 502, which has various components in electrical communication with each other using a connection 516, such as a bus, in accordance with some implementations. The example computing system architecture 501 includes a processor 506 that is in electrical communication with various system components, using the connection 516, and including the system memory 505. In some embodiments, the system memory 505 includes read-only memory (ROM), random-access memory (RAM), and other such memory technologies including, but not limited to, those described herein. In some embodiments, the example computing system architecture 501 includes a cache 503 of high-speed memory connected directly with, in close proximity to, or integrated as part of the processor 506. The system architecture 501 can copy data from the memory 505 and/or the storage device 517 to the cache 503 for quick access by the processor 506. In this way, the cache 503 can provide a performance boost that decreases or eliminates processor delays in the processor 506 due to waiting for data. Using modules, methods, and services such as those described herein, the processor 506 can be configured to perform various actions. In some embodiments, the cache 503 may include multiple types of cache including, for example, level one (L1) and level two (L2) cache. The memory 505 may be referred to herein as system memory or computer system memory. The memory 505 may include, at various times, elements of an operating system, one or more applications, data associated with the operating system or the one or more applications, or other such data associated with the computing device 502.

Other system memory 505 can be available for use as well. The memory 505 can include multiple different types of memory with different performance characteristics. The processor 506 can include any general-purpose processor and one or more hardware or software services, such as service 504 stored in storage device 517, configured to control the processor 506 as well as a special-purpose processor where software instructions are incorporated into the actual processor design. The processor 506 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 506 can be a microprocessor, a microcontroller, a digital signal processor (“DSP”), or a combination of these and/or other types of processors. In some embodiments, the processor 506 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units.

To enable user interaction with the computing system architecture 501, an input device 507 can represent any number of input mechanisms, such as a microphone for speech, a touch-sensitive screen for gesture or graphical input, keyboard, mouse, motion input, pen, and other such input devices. An output device 508 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 501. In some embodiments, the input device 507 and/or the output device 508 can be coupled to the computing device 502 using a remote connection device such as, for example, a communication interface such as the network interface 509 described herein. In such embodiments, the communication interface can govern and manage the input and output received from the attached input device 507 and/or output device 508. As may be contemplated, there is no restriction on operating on any particular hardware arrangement and accordingly the basic features here may easily be substituted for other hardware, software, or firmware arrangements as they are developed.

In some embodiments, the storage device 517 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 517 can include hardware and/or software services such as service 504 that can control or configure the processor 506 to perform one or more functions including, but not limited to, the methods, processes, functions, systems, and services described herein in various embodiments. In some embodiments, the hardware or software services can be implemented as modules. As illustrated in example computing system architecture 501, the storage device 517 can be connected to other parts of the computing device 502 using the system connection 516. In some embodiments, a hardware service or hardware module such as service 504, that performs a function can include a software component stored in a non-transitory computer-readable medium that, in connection with the necessary hardware components, such as the processor 506, connection 516, cache 503, storage device 517, memory 505, input device 507, output device 508, and so forth, can carry out the functions such as those described herein.

The disclosed systems and service of a the integrated service (e.g., integrated service 102 described herein at least in connection with FIG. 1) can be performed using a computing system such as the example computing system illustrated in FIG. 5, using one or more components of the example computing system 100 (e.g., an architecture of the computing system 100). An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and systems for facilitating requests using an integrated service (e.g., integrated service 102 described herein at least in connection with FIG. 1) described herein by, for example, executing code using a processor such as processor of the user device 104 wherein the code is stored in memory such as memory 505 as described herein. One or more of a user device, a provider server or system, a database system, or other such devices, services, or systems may include some or all of the components of the computing system such as the example computing system illustrated in FIG. 5, using one or more components of the example computing system architecture 501 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital representative (PDA), a server, or a combination of two or more of these. Where appropriate, the computer system may include one or more computer systems; be unitary or distributed; span multiple locations; span multiple machines; and/or reside in a cloud computing system which may include one or more cloud components in one or more networks as described herein in association with the computing resources provider 513. Where appropriate, one or more computer systems may perform without substantial spatial or temporal limitation one or more steps of one or more methods described or illustrated herein. As an example, and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 506 can be a conventional microprocessor such as an Intel® microprocessor, an AMD® microprocessor, a Motorola® microprocessor, or other such microprocessors. One of skill in the relevant art will recognize that the terms “machine-readable (storage) medium” or “computer-readable (storage) medium” include any type of device that is accessible by the processor.

The memory 505 can be coupled to the processor 506 by, for example, a connector such as connection 516, or a bus. As used herein, a connector or bus such as connection 516 is a communications system that transfers data between components within the computing device 502 and may, in some embodiments, be used to transfer data between computing devices. The connection 516 can be a data bus, a memory bus, a system bus, or other such data transfer mechanism. Examples of such connectors include, but are not limited to, an industry standard architecture (ISA″ bus, an extended ISA (EISA) bus, a parallel AT attachment (PATA″ bus (e.g., an integrated drive electronics (IDE) or an extended IDE (EIDE) bus), or the various types of parallel component interconnect (PCI) buses (e.g., PCI, PCIe, PCI-104, etc.).

The memory 505 can include RAM including, but not limited to, dynamic RAM (DRAM), static RAM (SRAM), synchronous dynamic RAM (SDRAM), non-volatile random-access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 505 can also include magnetic or optical data storage media including read-only (e.g., CD ROM and DVD ROM) or otherwise (e.g., CD or DVD). The memory can be local, remote, or distributed.

As described above, the connection 516 (or bus) can also couple the processor 506 to the storage device 517, which may include non-volatile memory or storage, and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data. Some of this data may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 517. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 516 can also couple the processor 506 to a network interface device such as the network interface 509. The interface can include one or more of a modem or other such network interfaces including, but not limited to those described herein. It will be appreciated that the network interface 509 may be considered to be part of the computing device 502 or may be separate from the computing device 502. The network interface 509 can include one or more of an analog modem, Integrated Services Digital Network (ISDN) modem, cable modem, token ring interface, satellite transmission interface, or other interfaces for coupling a computer system to other computer systems. In some embodiments, the network interface 509 can include one or more input and/or output (I/O) devices. The I/O devices can include, by way of example but not limitation, input devices such as input device 507 and/or output devices such as output device 508. For example, the network interface 509 may include a keyboard, a mouse, a printer, a scanner, a display device, and other such components. Other examples of input devices and output devices are described herein. In some embodiments, a communication interface device can be implemented as a complete and separate computing device.

In operation, the computer system can be controlled by operating system software that includes a file management system, such as a disk operating system. One example of operating system software with associated file management system software is the family of Windows® operating systems and their associated file management systems. Another example of operating system software with its associated file management system software is the Linux™ operating system and its associated file management system including, but not limited to, the various types and implementations of the Linux® operating system and their associated file management systems. The file management system can be stored in the non-volatile memory and/or drive unit and can cause the processor to execute the various acts required by the operating system to input and output data and to store data in the memory, including storing files on the non-volatile memory and/or drive unit. As may be contemplated, other types of operating systems such as, for example, MacOS®, other types of UNIX® operating systems (e.g., BSD™ and descendants, Xenix™, SunOS™, HP-UX®, etc.), mobile operating systems (e.g., iOS® and variants, Chrome®, Ubuntu Touch®, watchOS®, Windows 10 Mobile®, the Blackberry® OS, etc.), and real-time operating systems (e.g., VxWorks®, QNX®, eCos®, RTLinux®, etc.) may be considered as within the scope of the present disclosure. As may be contemplated, the names of operating systems, mobile operating systems, real-time operating systems, languages, and devices, listed herein may be registered trademarks, service marks, or designs of various associated entities.

In some embodiments, the computing device 502 can be connected to one or more additional computing devices such as computing device 511 via a network 510 using a connection such as the network interface 509. In such embodiments, the computing device 511 may execute one or more services 512 to perform one or more functions under the control of, or on behalf of, programs and/or services operating on computing device 502. In some embodiments, a computing device such as computing device 511 may include one or more of the types of components as described in connection with computing device 502 including, but not limited to, a processor such as processor 506, a connection such as connection 516, a cache such as cache 503, a storage device such as storage device 517, memory such as memory 505, an input device such as input device 507, and an output device such as output device 508. In such embodiments, the computing device 511 can carry out the functions such as those described herein in connection with computing device 502. In some embodiments, the computing device 502 can be connected to a plurality of computing devices such as computing device 511, each of which may also be connected to a plurality of computing devices such as computing device 511. Such an embodiment may be referred to herein as a distributed computing environment.

The network 510 can be any network including an internet, an intranet, an extranet, a cellular network, a Wi-Fi network, a local area network (LAN), a wide area network (WAN), a satellite network, a Bluetooth® network, a virtual private network (VPN), a public switched telephone network, an infrared (IR) network, an internet of things (IoT network) or any other such network or combination of networks. Communications via the network 510 can be wired connections, wireless connections, or combinations thereof. Communications via the network 510 can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), and other such communications protocols.

Communications over the network 510, within the computing device 502, within the computing device 511, or within the computing resources provider 513 can include information, which also may be referred to herein as content. The information may include text, graphics, audio, video, haptics, and/or any other information that can be provided to a user of the computing device such as the computing device 502. In some embodiments, the information can be delivered using a transfer protocol such as Hypertext Markup Language (HTML), Extensible Markup Language (XML), JavaScript®, Cascading Style Sheets (CSS), JavaScript® Object Notation (JSON), and other such protocols and/or structured languages. The information may first be processed by the computing device 502 and presented to a user of the computing device 502 using forms that are perceptible via sight, sound, smell, taste, touch, or other such mechanisms. In some embodiments, communications over the network 510 can be received and/or processed by a computing device configured as a server. Such communications can be sent and received using PHP: Hypertext Preprocessor (“PHP”), Python™, Ruby, Perl® and variants, Java®, HTML, XML, or another such server-side processing language.

In some embodiments, the computing device 502 and/or the computing device 511 can be connected to a computing resources provider 513 via the network 510 using a network interface such as those described herein (e.g., network interface 509). In such embodiments, one or more systems (e.g., service 514 and service 515) hosted within the computing resources provider 513 (also referred to herein as within “a computing resources provider environment”) may execute one or more services to perform one or more functions under the control of, or on behalf of, programs and/or services operating on computing device 502 and/or computing device 511. Systems such as service 514 and service 515 may include one or more computing devices such as those described herein to execute computer code to perform the one or more functions under the control of, or on behalf of, programs and/or services operating on computing device 502 and/or computing device 511.

For example, the computing resources provider 513 may provide a service, operating on service 514 to store data for the computing device 502 when, for example, the amount of data that the computing device 502 exceeds the capacity of storage device 517. In another example, the computing resources provider 513 may provide a service to first instantiate a virtual machine (VM) on service 515, use that VM to access the data stored on service 515, perform one or more operations on that data, and provide a result of those one or more operations to the computing device 502. Such operations (e.g., data storage and VM instantiation) may be referred to herein as operating “in the cloud,” “within a cloud computing environment,” or “within a hosted virtual machine environment,” and the computing resources provider 513 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc.

Services provided by a computing resources provider 513 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, server-less hosting, virtual reality (VR) systems, and augmented reality (AR) systems. Various techniques to facilitate such services include, but are not limited to, virtual machines, virtual storage, database services, system schedulers (e.g., hypervisors), resource management systems, various types of short-term, mid-term, long-term, and archival storage devices, etc.

As may be contemplated, the systems such as service 514 and service 515 may implement versions of various services (e.g., the service 504 or the service 512) on behalf of, or under the control of, computing device 502 and/or computing device 511. Such implemented versions of various services may involve one or more virtualization techniques so that, for example, it may appear to a user of computing device 502 that the service 504 is executing on the computing device 502 when the service is executing on, for example, service 514. As may also be contemplated, the various services operating within the computing resources provider 513 environment may be distributed among various systems within the environment as well as partially distributed onto computing device 511 and/or computing device 502.

The following examples illustrate various aspects of the present disclosure. As used below, any reference to a series of examples is to be understood as a reference to each of those examples disjunctively (e.g., “Examples 1-4” is to be understood as “Examples 1, 2, 4, or 4”).
Example 1 is a computer-implemented method, comprising: receiving a request to initiate a secured transaction associated with a patient account, wherein the request is received by a server associated with an integrated service, and wherein a plurality of instances of the request are performed by the server simultaneously; generating one or more inquiries pertaining to the secured transaction, wherein the one or more inquiries are generated using the request, and wherein the one or more inquiries are requests for information used to process the secured transaction; transmitting, using a first custom application programming interface (API), a data request to a secured database for patient data associated with the secured transaction and the patient account; receiving, from the secured database using the first custom API, the patient data, wherein, the patient data includes secure private data associated with the secured transaction; populating a portion of the one or more inquiries by: generating a comparison of the one or more inquiries to the patient data; and identifying at least one matching inquiry based on the comparison, wherein the at least one matching inquiry indicates that an element of patient data corresponds to an inquiry from the one or more inquiries; and facilitating presentation of the one or more inquiries on a user interface of a display device, wherein the portion of the one or more inquiries are distinguished upon presentation by populating the portion of the one or more inquiries with respective elements of patient data.
Example 2 is the computer-implemented method of example(s) 1, further comprising: receiving input responsive to the one or more inquiries; transmitting an action request associated with the secured transaction, wherein the action request includes the input responsive to the one or more inquiries and the element of the patient data responsive to the at least one matching inquiry, wherein when the action request is received by a transaction manager, the transaction manager forwards the action request to a payment platform associated with the action request in accordance with the input and the element of the patient data;
   receiving a response associated with the action request; and presenting the response using a second custom GUI.
Example 3 is the computer-implemented method of example(s) 1-2, further comprising: receiving input responsive to the one or more inquiries; and transmitting a credit request associated with the secured transaction, wherein the credit request includes the input responsive to the one or more inquiries and the element of the patient data responsive to the at least one matching inquiry, wherein when the credit request is received by a transaction manager, the transaction manager forwards the credit request to a financial institution associated with the credit request in accordance with the input and the element of the patient data; receiving a response associated with the credit request; and presenting the response using a second custom GUI. Example 4 is the computer-implemented method of example(s) 1-3, further comprising: receiving input responsive to the one or more inquiries; and outputting a payment request associated with the secured transaction, wherein the payment request includes the input responsive to the one or more inquiries and the element of the patient data responsive to the at least one matching inquiry. Example 5 is the computer-implemented method of example(s) 1-4, wherein: the request is transmitted by a plug-in operating from an independent software module operating on a user device; and the computer-implemented method further comprises: transmitting a notification to request transaction data associated with the secured transaction, wherein when the independent software module receives the notification, the independent software module transmits a transaction data request; and transmitting data responsive to the transaction data request, wherein when received by the independent software module, the independent software module updates one or more local databases associated with the user device with the data.
Example 6 is the computer-implemented method of example(s) 1-5, further comprising: outputting a status request, wherein when the status request is received by a transaction manager, the transaction manager provides a status associated with the secured transaction.
Example 7 is the computer-implemented method of example(s) 1-6, further comprising: outputting a refund request, wherein the refund request includes a completed transaction and patient data associated with the completed transaction; receiving a response associated with the refund request, wherein the response includes at least one of an approval or a denial of a refund associated with the completed transaction; and outputting the response. Example 8 is the computer-implemented method of example(s) 1-7, wherein when the one or more inquiries are received by a user device, the user device transmits the one or more inquiries.
Example 9 is the computer-implemented method of example(s) 1-8, wherein the display device is a patient device associated with the patient data, and wherein inputs responsive to the one or more inquiries are received through the patient device.
Example 10 is the computer-implemented method of example(s) 1-9, further comprising: outputting an additional patient data request; and receiving additional patient data in response to the additional patient data request, wherein the additional patient data is received from the secured database, and wherein the patient data includes at least identifying information and historical completed transaction.
Example 11 is the computer-implemented method of example(s) 1-10, wherein: the display device is associated with a primary location; and the computer-implemented method further comprises: receiving one or more operational settings associated with the integrated service; and installing the one or more operational settings throughout a network of secondary user devices associated with the primary location, wherein the secondary user devices are unable to alter the one or more operational settings.
Example 12 is the computer-implemented method of example(s) 1-11, further comprising: receiving one or more software updates associated with the integrated service; and automatically installing the one or more software updates on the display device.
Example 13 is the computer-implemented method of example(s) 1-12, wherein identifying the matching inquiry of the one or more inquiries is performed by a machine-learning model.
Example 14 is the computer-implemented method of example(s) 1-13, further comprising: updating the secured database with data pertaining to the secured transaction.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a keypad, a touch interface, a microphone, a camera, and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 502) include, but is not limited to, desktop computers, laptop computers, server computers, hand-held computers, tablets, smart phones, personal digital representatives, digital home representatives, wearable devices, smart devices, and combinations of these and/or other such computing devices as well as machines and apparatuses in which a computing device has been incorporated and/or virtually implemented.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as that described herein. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general-purpose processor may be a microprocessor; but in the alternative, the processor may be any conventional processor, controller, microcontroller, or state machine. A processor may also be implemented as a combination of computing devices (e.g., a combination of a DSP and a microprocessor), a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

As used herein, the term “machine-readable media” and equivalent terms “machine-readable storage media,” “computer-readable media,” and “computer-readable storage media” refer to media that includes, but is not limited to, portable or non-portable storage devices, optical storage devices, removable or non-removable storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), solid state drives (SSD), flash memory, memory, or memory devices.

A machine-readable medium or machine-readable storage medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like. Further examples of machine-readable storage media, machine-readable media, or computer-readable (storage) media include but are not limited to recordable type media such as volatile and non-volatile memory devices, floppy and other removable disks, hard disk drives, optical disks (e.g., CDs, DVDs, etc.), among others, and transmission type media such as digital and analog communication links.

As may be contemplated, while examples herein may illustrate or refer to a machine-readable medium or machine-readable storage medium as a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

Some portions of the detailed description herein may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

It is also noted that individual implementations may be described as a process which is depicted as a flowchart, a flow diagram, a data flow diagram, a structure diagram, or a block diagram (e.g., the example routine 400 of FIG. 4). Although a flowchart, a flow diagram, a data flow diagram, a structure diagram, or a block diagram may describe the operations as a sequential process, many of the operations can be performed in parallel or concurrently. In addition, the order of the operations may be re-arranged. A process illustrated in a figure is terminated when its operations are completed, but could have additional steps not included in the figure. A process may correspond to a method, a function, a procedure, a subroutine, a subprogram, etc. When a process corresponds to a function, its termination can correspond to a return of the function to the calling function or the main function.

In some embodiments, one or more implementations of an algorithm such as those described herein may be implemented using a machine learning or artificial intelligence algorithm. Such a machine learning or artificial intelligence algorithm may be trained using supervised, unsupervised, reinforcement, or other such training techniques. For example, a set of data may be analyzed using one of a variety of machine learning algorithms to identify correlations between different elements of the set of data without supervision and feedback (e.g., an unsupervised training technique). A machine learning data analysis algorithm may also be trained using sample or live data to identify potential correlations. Such algorithms may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, linear classification, artificial neural networks, anomaly detection, and such. More generally, machine learning or artificial intelligence methods may include regression analysis, dimensionality reduction, meta- learning, reinforcement learning, deep learning, and other such algorithms and/or methods. As may be contemplated, the terms “machine learning” and “artificial intelligence” are frequently used interchangeably due to the degree of overlap between these fields and many of the disclosed techniques and algorithms have similar approaches.

As an example of a supervised training technique, a set of data can be selected for training of the machine learning model to facilitate identification of correlations between members of the set of data. The machine learning model may be evaluated to determine, based on the sample inputs supplied to the machine learning model, whether the machine learning model is producing accurate correlations between members of the set of data. Based on this evaluation, the machine learning model may be modified to increase the likelihood of the machine learning model identifying the desired correlations. The machine learning model may further be dynamically trained by soliciting feedback from users of a system as to the efficacy of correlations provided by the machine learning algorithm or artificial intelligence algorithm (i.e., the supervision). The machine learning algorithm or artificial intelligence may use this feedback to improve the algorithm for generating correlations (e.g., the feedback may be used to further train the machine learning algorithm or artificial intelligence to provide more accurate correlations).

The various examples of flowcharts, flow diagrams, data flow diagrams, structure diagrams, or block diagrams discussed herein may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments) such as those described herein. A processor(s), implemented in an integrated circuit, may perform the necessary tasks.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

It should be noted, however, that the algorithms and displays presented herein are not inherently related to any particular computer or other apparatus. Various general-purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC (e.g., an iPad®, a Microsoft Surface®, a Chromebook®, etc.), a laptop computer, a set-top box (STB), a personal digital representative (PDA), a mobile device (e.g., a cellular telephone, an iPhone®, and Android® device, a Blackberry®, etc.), a wearable device, an embedded computer system, an electronic book reader, a processor, a telephone, a web appliance, a network router, switch or bridge, or any system capable of executing a set of instructions (sequential or otherwise) that specify actions to be taken by that system. The system may also be a virtual system such as a virtual version of one of the aforementioned devices that may be hosted on another computer device such as the computing device 502.

In general, the routines executed to implement the implementations of the disclosure, may be implemented as part of an operating system or a specific application, component, program, object, module, or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting or restricting the subject matter to the precise forms disclosed. Persons skilled in the relevant art can appreciate that many modifications and variations are possible in light of the above disclosure and may be made thereto without departing from the broader scope of the embodiments as set forth herein. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number, respectively. The word “or,” in reference to a list of two or more items, covers all of the following interpretations of the word: any of the items in the list, all of the items in the list, or any combination of the items in the list.

As used herein, the terms “a” and “an” and “the” and other such singular referents are to be construed to include both the singular and the plural, unless otherwise indicated herein or clearly contradicted by context.

As used herein, the terms “comprising,” “having,” “including,” and “containing” are to be construed as open-ended (e.g., “including” is to be construed as “including, but not limited to”), unless otherwise indicated or clearly contradicted by context.

As used herein, the recitation of ranges of values is intended to serve as a shorthand method of referring individually to each separate value falling within the range, unless otherwise indicated or clearly contradicted by context. Accordingly, each separate value of the range is incorporated into the specification as if it were individually recited herein.

As used herein, use of the terms “set” (e.g., “a set of items”) and “subset” (e.g., “a subset of the set of items”) is to be construed as a nonempty collection including one or more members unless otherwise indicated or clearly contradicted by context. Furthermore, unless otherwise indicated or clearly contradicted by context, the term “subset” of a corresponding set does not necessarily denote a proper subset of the corresponding set but that the subset and the set may include the same elements (i.e., the set and the subset may be the same).

As used herein, use of conjunctive language such as “at least one of A, B, and C” is to be construed as indicating one or more of A, B, and C (e.g., any one of the following nonempty subsets of the set {A, B, C}, namely: {A}, {B}, {C}, {A, B}, {A, C}, {B, C}, or {A, B, C}) unless otherwise indicated or clearly contradicted by context. Accordingly, conjunctive language such as “as least one of A, B, and C” does not imply a requirement for at least one of A, at least one of B, and at least one of C.

As used herein, the use of examples or exemplary language (e.g., “such as” or “as an example”) is intended to more clearly illustrate embodiments and does not impose a limitation on the scope unless otherwise claimed. Such language in the specification should not be construed as indicating any non-claimed element is required for the practice of the embodiments described and claimed in the present disclosure.

As used herein, where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 45 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods, and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules (e.g., structures or elements of a functional system), alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method, comprising:
receiving a request to initiate a secured transaction associated with a patient account, wherein the request is received by a server associated with an integrated service, and wherein a plurality of instances of the request are performed by the server simultaneously;
generating one or more inquiries pertaining to the secured transaction, wherein the one or more inquiries are generated using the request, and wherein the one or more inquiries are requests for information used to process the secured transaction;
transmitting, using a first custom application programming interface (API), a data request to a secured database for patient data associated with the secured transaction and the patient account;
receiving, from the secured database using the first custom API, the patient data, wherein, the patient data includes secure private data associated with the secured transaction;
populating a portion of the one or more inquiries by:
generating a comparison of the one or more inquiries to the patient data; and
identifying at least one matching inquiry based on the comparison, wherein the at least one matching inquiry indicates that an element of patient data corresponds to an inquiry from the one or more inquiries; and
facilitating presentation of the one or more inquiries on a user interface of a display device, wherein the portion of the one or more inquiries are distinguished upon presentation by populating the portion of the one or more inquiries with respective elements of patient data.

2. The computer-implemented method of claim 1, further comprising:
receiving input responsive to the one or more inquiries;
transmitting an action request associated with the secured transaction, wherein the action request includes the input responsive to the one or more inquiries and the element of the patient data responsive to the at least one matching inquiry, wherein when the action request is received by a transaction manager, the transaction manager forwards the action request to a payment platform associated with the action request in accordance with the input and the element of the patient data;
receiving a response associated with the action request; and
presenting the response using a second custom GUI.

3. The computer-implemented method of claim 1, further comprising:
receiving input responsive to the one or more inquiries;
transmitting a credit request associated with the secured transaction, wherein the credit request includes the input responsive to the one or more inquiries and the element of the patient data responsive to the at least one matching inquiry, wherein when the credit request is received by a transaction manager, the transaction manager forwards the credit request to a financial institution associated with the credit request in accordance with the input and the element of the patient data;
receiving a response associated with the credit request; and
presenting the response using a second custom GUI.

4. The computer-implemented method of claim 1, further comprising:
receiving input responsive to the one or more inquiries; and
outputting a payment request associated with the secured transaction, wherein the payment request includes the input responsive to the one or more inquiries and the element of the patient data responsive to the at least one matching inquiry.

5. The computer-implemented method of claim 1, wherein:
the request is transmitted by a plug-in operating from an independent software module operating on a user device; and
the computer-implemented method further comprises:
transmitting a notification to request transaction data associated with the secured transaction, wherein when the independent software module receives the notification, the independent software module transmits a transaction data request; and
transmitting data responsive to the transaction data request, wherein when received by the independent software module, the independent software module updates one or more local databases associated with the user device with the data.

6. The computer-implemented method of claim 1, further comprising:
outputting a status request, wherein when the status request is received by a transaction manager, the transaction manager provides a status associated with the secured transaction.

7. The computer-implemented method of claim 1, further comprising:
outputting a refund request, wherein the refund request includes a completed transaction and patient data associated with the completed transaction;
receiving a response associated with the refund request, wherein the response includes at least one of an approval or a denial of a refund associated with the completed transaction; and
outputting the response.

8. The computer-implemented method of claim 1, wherein when the one or more inquiries are received by a user device, the user device transmits the one or more inquiries.

9. The computer-implemented method of claim 1, wherein the display device is a patient device associated with the patient data, and wherein inputs responsive to the one or more inquiries are received through the patient device.

10. The computer-implemented method of claim 1, further comprising:
outputting an additional patient data request; and
receiving additional patient data in response to the additional patient data request, wherein the additional patient data is received from the secured database, and wherein the patient data includes at least identifying information and historical completed transaction.

11. The computer-implemented method of claim 1, wherein:
the display device is associated with a primary location; and
the computer-implemented method further comprises:
receiving one or more operational settings associated with the integrated service; and
installing the one or more operational settings throughout a network of secondary user devices associated with the primary location, wherein the secondary user devices are unable to alter the one or more operational settings.

12. The computer-implemented method of claim 1, further comprising:
receiving one or more software updates associated with the integrated service; and
automatically installing the one or more software updates on the display device.

13. The computer-implemented method of claim 1, wherein identifying the at least one matching inquiry of the one or more inquiries is performed by a machine-learning model.

14. The computer-implemented method of claim 1, further comprising:
updating the secured database with data pertaining to the secured transaction.

15. A system comprising:
one or more processors and a non-transitory computer-readable medium storing instructions that, when executed by the one or more processors, cause the one or more processors to perform operations comprising:
receiving a request to initiate a secured transaction associated with a patient account, wherein the request is received by a server associated with an integrated service, and wherein a plurality of instances of the request are performed by the server simultaneously;
generating one or more inquiries pertaining to the secured transaction, wherein the one or more inquiries are generated using the request, and wherein the one or more inquiries are requests for information used to process the secured transaction;
transmitting, using a first custom application programming interface (API), a data request to a secured database for patient data associated with the secured transaction and the patient account;
receiving, from the secured database using the first custom API, the patient data, wherein, the patient data includes secure private data associated with the secured transaction;
populating a portion of the one or more inquiries by:
generating a comparison of the one or more inquiries to the patient data; and
identifying at least one matching inquiry based on the comparison, wherein the at least one matching inquiry indicates that an element of patient data corresponds to an inquiry from the one or more inquiries; and
facilitating presentation of the one or more inquiries on a user interface of a display device, wherein the portion of the one or more inquiries are distinguished upon presentation by populating the portion of the one or more inquiries with respective elements of patient data.

16. The system of claim 15, wherein the instructions further cause the one or more processors to perform operations comprising:
receiving input responsive to the one or more inquiries;
transmitting an action request associated with the secured transaction, wherein the action request includes the input responsive to the one or more inquiries and the element of the patient data responsive to the at least one matching inquiry, wherein when the action request is received by a transaction manager, the transaction manager forwards the action request to a platform associated with the action request in accordance with the input and the element of the patient data;
receiving a response associated with the action request; and
presenting the response using a second custom GUI.

17. The system of claim 15, wherein the instructions further cause the one or more processors to perform operations comprising:
receiving input responsive to the one or more inquiries;
transmitting a credit request associated with the secured transaction, wherein the credit request includes the input responsive to the one or more inquiries and the element of the patient data responsive to the at least one matching inquiry, wherein when the credit request is received by a transaction manager, the transaction manager forwards the credit request to a financial institution associated with the credit request in accordance with the input and the element of the patient data;
receiving a response associated with the credit request; and
presenting the response using a second custom GUI.

18. The system of claim 15, wherein the instructions further cause the one or more processors to perform operations comprising:
receiving input responsive to the one or more inquiries; and
outputting a payment request associated with the secured transaction, wherein the payment request includes the input responsive to the one or more inquiries and the element of the patient data responsive to the at least one matching inquiry.

19. The system of claim 15, wherein:
the request is transmitted by a plug-in operating from an independent software module operating on a user device; and
the instructions further cause the one or more processors to perform operations comprising:
transmitting a notification to request transaction data associated with the secured transaction, wherein when the independent software module receives the notification, the independent software module transmits a transaction data request; and
transmitting data responsive to the transaction data request, wherein when received by the independent software module, the independent software module updates one or more local databases associated with the user device with the data.

20. A non-transitory computer-readable medium storing instructions that when executed by one or more processors cause the processors to perform operations comprising:
receiving a request to initiate a secured transaction associated with a patient account, wherein the request is received by a server associated with an integrated service, and wherein a plurality of instances of the request are performed by the server simultaneously;
generating one or more inquiries pertaining to the secured transaction, wherein the one or more inquiries are generated using the request, and wherein the one or more inquiries are requests for information used to process the secured transaction;
transmitting, using a first custom application programming interface (API), a data request to a secured database for patient data associated with the secured transaction and the patient account;
receiving, from the secured database using the first custom API, the patient data, wherein, the patient data includes secure private data associated with the secured transaction;
populating a portion of the one or more inquiries by:
generating a comparison of the one or more inquiries to the patient data; and
identifying at least one matching inquiry based on the comparison, wherein the at least one matching inquiry indicates that an element of patient data corresponds to an inquiry from the one or more inquiries; and
facilitating presentation of the one or more inquiries on a user interface of a display device, wherein the portion of the one or more inquiries are distinguished upon presentation by populating the portion of the one or more inquiries with respective elements of patient data.
